(19) United States
(12) Patent Application Publication
(10) Pub. No.: US 2001/0055541 A1
(43) Pub. Date: Dec. 27, 2001

(54) Porous material disinfection method

(76) Inventor: Durand M. Smith, Placitas, NM (US)

Correspondence Address: TRASK BRITT, P.O. BOX 2550, SALT LAKE CITY, UT, 84110, US

(21) Appl. No.: 09/759,888

(22) Filed: Jan. 12, 2001

Related U.S. Application Data
(60) Provisional application No. 60/176,333, filed on Jan. 14, 2000.

(51) Int. Cl. A61L 2/20

(52) U.S. Cl. 422/28

(57) ABSTRACT

The invention includes a method of reducing the microbial population on a item comprising, in whole or in part, porous material. The method includes contacting the porous portion of the item with both a surfactant and gaseous ozone, preferably in a sealed container.

### CROSS-REFERENCE TO RELATED APPLICATION

Under the provisions of 35 U.S.C. § 1.19(e), priority is claimed from U.S. Provisional Patent Application Ser. No. 60/176,333 filed Jan. 14, 2000.

### BACKGROUND OF THE INVENTION

1. Technical Field

The invention relates generally to a method of reducing the microbial load on porous materials. More specifically, the invention relates to a method of disinfecting, sanitizing, or sterilizing porous materials involving the use of ozone together with a surfactant.

2. Background

Items made from porous materials, such as sponges, have a wide variety of uses. Sponges are especially useful in the cleaning field. For instance, sponges are used in homes, restaurants, garages, car washes, and hospitals. Their absorbency makes them ideal for cleaning and soaking up liquids such as dishwater, spills, and, in the hospital, blood and other bodily fluids.

Unfortunately, due to the inherent structure of porous materials, such as sponges, although they are useful for cleaning, they themselves are very difficult to clean or disinfect. The inherent cell structure of the sponge makes it difficult to apply liquid disinfectant to especially the inner cells. This, taken together with the fact the cell structure also greatly increases surface area, provides many growth surfaces for bacteria, fungus and other microbes.

In the home, this unchecked microbial growth can cause odor problems. In restaurants, it may lead to health problems if certain microbes are allowed to grow and spread to utensils, cutlery, and eventually food. In hospitals, the potential problems with blood-borne organisms such as hepatitis or human immunodeficiency virus are vast.

In disinfecting other items, such as hard surfaces, ozone has found use as a disinfectant. See, e.g., International Patent Application No. PCT/US94/06463 to Hei et al. of Ecolab, Inc. (Mar. 5, 1995) and U.S. Pat. Nos. 5,484,549, 5,567,444, and 5,858,443 also to Hei et al.

Ozone is an unstable triatomic allotrope of oxygen. It is produced in an energized environment wherein molecular oxygen dissociates into monatomic oxygen which subsequently collides and recombines with an oxygen molecule to form a highly reactive ozone molecule.

Cleaning of hard surfaces (e.g., hard surface engineering material including glass, metals including stainless steel, steel, aluminum, and synthetic substances such as acrylic plastics, epoxy, polyimide condensation products according to the aforementioned Hei et al. patent publications) enjoys the advantage, in comparison to cleaning or disinfecting porous materials, of having a relatively impermeable, smooth, open surface to work on.

Presently, for porous materials such as artificial or natural materials or filter materials, the most commonly employed method of addressing the growth of microbes and the associated odor and health concerns is to dispose of them. Disposal obviously facilitates containment of blood-borne pathogens as well as other infectious agents. However, reuse of these items could constitute an immense savings in capital expenditures regarding purchase of single use items and their bio-hazardous disposal costs, as well as demonstrating environmental responsibility. In this environment, the necessity for a reliable means of reducing the bio-burden to acceptable levels is of critical importance.

It would be an improvement in the art to have a relatively economical, reliable method of disinfecting, sanitizing or sterilizing items containing porous materials such as sponges.

### BRIEF SUMMARY OF THE INVENTION

The invention includes a method of reducing the microbial load on an item that is partially or completely comprised of a porous material. The method includes applying a surfactant to the item (e.g., by manually working it in) and placing it into a chamber into which ozone is introduced, thus bringing the porous material into contact with the surfactant and ozone.

The process results in disinfected, sanitized, deodorized and/or sterilized porous materials. Due to ozone's higher oxidation potential, the duration of the treatment can be reduced relative to those cycles using only ozone or only surfactant. This contributes to increased porous material life by decreasing stress on the porous material through limited mechanical action and the use of room temperature.

### BRIEF DESCRIPTION OF THE SEVERAL VIEWS OF THE DRAWINGS

FIG. 1 graphically depicts a chamber useful for carrying out the invention.

FIG. 2 is a graph depicting the results of EXAMPLE II.

### DETAILED DESCRIPTION OF THE INVENTION

The most prominent features of ozone as a biocide lie in its speed and selectivity in oxidation. Biocidal effects are believed to primarily be achieved through oxidation. Consistent with this belief, the ability of any chemical to reduce microbial viability is in direct proportion to its oxidation potential. Ozone is a very powerful oxidizing agent.

Disinfection with the use of ozone may proceed by oxidation directly and by intermediate hydroperoxy compounds that can interact with cytosolic components. Organic ozone chemistry would predict that oxidized organic compounds containing carbon-carbon double bonds give rise to hydroperoxyalcohols. Evidence exists that organic peroxides exert a stronger bactericidal action than hydrogen peroxide due to a greater tendency to decompose. No evidence is believed to exist in the literature of any microorganism that is resistant to the effects of ozone exposure.

In addition to demonstrating powerful capabilities in the destruction or inactivation of bacteria, fungi and protozoa, ozone has been shown to be virucidal. The efficacy of ozone has been reported to range from (all of the following values given reported a 99% reduction) 2.2 milligrams per liter (mg/l) for Escherichia coli in 19 minutes from raw waste water; 0.02 mg/l for Candida tropicalis in 0.30 minutes from ozone-demand free water; 1.2 mg/l for Naegleria gruberi in 1.1 minutes from ozone-demand free phosphate buffer solution and 0.2 mg/l for Poliovirus type I in 9 minutes from activated sludge effluent. With regard to bacterial spores (specifically, Bacillus subtilis v. globigii), ozone has been shown to achieve a four-log reduction within 1.5-2 minutes when water is purged with 3% ozone by weight. Using a non-toxic concentration of 4 micrograms (μg) ozone per milliliter (ml) of serum, ozone can achieve a six-log reduction in the infectious titer of human immunodeficiency virus (“HIV”).

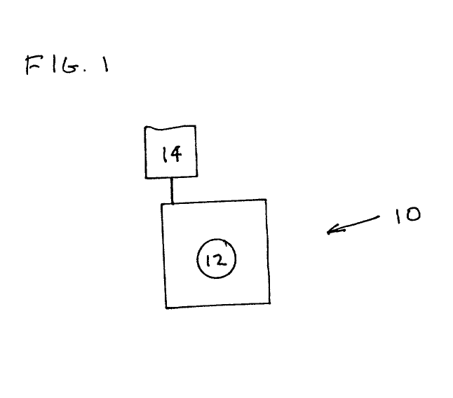

Although not per se forming part of the instant invention, as depicted in FIG. 1, a gas tight chamber, generally 10, is useful in practicing the herein described methods. A chamber for use with the invention can be of any size sufficient to accommodate the porous items to be disinfected. For instance, for use with a single dishwashing sponge 12, the interior of the chamber could be, for example, about 20 cm by 10 cm by 20 cm. Larger chambers can be made for disinfecting multiple sponges, for instance in a restaurant or hospital setting.

The chamber preferably has a door or other means for allowing easy access to its interior for placement and eventual removal of an item of the porous material to be disinfected.

The chamber receives a stream of ozone, mixed with another gas, such as air. The flow of ozone into the chamber is preferably guided by a system of operation that uses electro-mechanical devices that are directed by electro-mechanical controls, under program control via a microprocessor, in order to deliver the correct concentrations and volume amounts of ozone gas, as well as optionally control and monitor temperature and time of various cycles.

An ozone generator 14 is in fluid communication with the interior of the chamber which contains the sponge 12. The ozone generator 14 may be one of a number of devices for generating ozone from air, oxygen or air enriched with oxygen, and its required capacity depends on the number and size of the chambers installed in a facility. Known ozone generators are disclosed in U.S. Pat. No. 5,145,350 to Dawson, and U.S. Pat. Nos. 1,096,991 to Blanchard, May 19, 1914; 3,836,786 to Lowther, Sep. 17, 1974; 3,891,561 to Lowther, Jun. 24, 1975; 3,899,683 to Lowther, Aug. 12, 1975; 3,903,426 to Lowther, Sep. 2, 1975; 3,954,586 to Lowther, May 4, 1976; 3,984,697 to Lowther, Oct. 5, 1976; 3,996,474 to Lowther, Dec. 7, 1976; 4,013,567 to Emelyanov et al., Mar. 22, 1977; 4,141,686 to Lewis, Feb. 27, 1979; 4,255,663 to Lewis, Mar. 10, 1981; 4,411,756 to Bennett et al., Oct. 25, 1983; 4,504,446 to Kunicki et al., Mar. 12, 1985; 4,780,277 to Tanaka et al., Oct. 25, 1988; 4,917,586 to Jacob, Apr. 17, 1990; 4,954,321 to Jensen, Sep. 4, 1990; 5,004,587 to Tacchi, Apr. 2, 1991; 5,089,098 to Tacchi, Feb. 18, 1992; 5,154,895 to Moon, Oct. 13, 1992; 5,211,919 to Conrad, May 18, 1993; 5,302,343 to Jacob, Apr. 12, 1994; 5,306,471 to Harbert et al., Apr. 26, 1994; and 5,433,927 to Mausgrover et al., Jul. 18, 1995, the disclosures of each of which are hereby incorporated by this reference in their entireties.

In general, the illustrated ozone generator 14 generates ozone by passing dry air or oxygen through a corona discharge produced by a high voltage at a high frequency which is applied to coaxial electrodes (not shown) within the generator 14. The electrode voltage is derived from an appropriate power supply. The temperature of the generator 14 may be controlled by cycling cooling water through the electrodes. In one embodiment, the ozone gas is generated from oxygen or oxygen-enriched air by a corona discharge device that produces concentrations ranging between about 1% to about 15% by weight of ozone.

The ozone output of the generator 14 can be controlled by the main controller by an appropriate (e.g., analog) signal level therefrom. In general, the ozone output is controlled by controlling the frequency of the ozone generating power applied to the electrodes of the generator 14. The ozone output of the generator 14 may also be varied by controlling the voltage level of the applied power. The illustrated ozone generator 14 may be controlled to vary its ozone output from about ten percent to full rated output. The main controller also has the capability of completely disabling the ozone generator 14 to entirely shut down the generation of ozone.

The ozone generator 14 may receive air through an air preparation unit (not shown) which filters and dries the air and regulates the pressure thereof. An air preparation unit preferably receives compressed air from an air compressor at an appropriate flow rate and pressure. A supply conduit from the compressor connects to a multiple stage pre-filter through a manually operated ball valve. A first pressure regulator regulates air pressure to a T-connector. A branch conduit from the connector leads to a second regulator, a lubricator, and an auxiliary conduit which connects to the main panel. The T-connector also connects to a coalescing filter and a twin regenerative air dryer which removes moisture from the compressed air fed to the ozone generator. From the air dryer, the air passes through a general purpose filter and a third regulator to provide air at an appropriate pressure to a main air supply conduit which supplies air to the ozone generator 14.

An ozone distribution or main panel includes a panel wall supporting an ozone distribution manifold which receives ozonated compressed air from a main ozone supply conduit and makes it available to a plurality of manifold outlet conduits. Each outlet conduit forms an ozone distribution circuit between the panel and a chamber.

Preferably, virtually all of the ozone injected into a chamber is consumed during the procedure. Thus, the detection of excessive levels of ambient ozone usually implies a leak in the plumbing between the distribution panel and the chamber. The optional placement of an ozone monitor or monitors (not shown) on the chamber is generally determined by the size and airflow patterns therein. These monitors inform the user in the unlikely event that an ozone leak should occur.

Preferred surfactants for use with the invention are non-cationic surfactants (e.g, nonionic, anionic, or mixtures thereof). Such surfactants are well known to those of skill in the art. A highly preferred surfactant for use with the invention is DAWN™ dishwashing detergent (readily commercially available from Procter & Gamble, Cincinnati, Ohio, US) which contains biodegradable anionic and nonionic surfactants with no phosphates. Other suitable surfactants include POWER PLUS DAWN™, SPECIAL CARE DAWN™, LEMON DAWN™, ULTRA DAWN™, ANTIBACTERIAL DAWN™, and MOUNTAIN SPRING DAWN™ dishwashing detergents (all readily commercially available from Procter & Gamble, Cincinnati, Ohio, US). Concentrations of greater than 1% are preferred. Residual DAWN™ left on the sponge after rinsing may be used.

The invention is further explained by the following illustrative EXAMPLES:

### EXAMPLES

### Example I

A protocol was developed to determine the resistance of various organisms to sterilization utilizing ozone gas. Six organisms were identified as being useful in determining the efficacy of ozone gas as a biocide. The data generated was used to determine the most resistant organism to ozone exposure. The six organisms listed here are commonly available and can be tested without significant precautions for the protection of personnel from pathogenic microorganisms.

The intent was to expose these six organisms to the level of ozone used in a medical sterilization device and thereby decipher which is the most resistant organism as demonstrated by its ability to maintain viability. The organism which endures ozone exposure the longest without becoming sterilized (sterility is defined as completely dead, rendered unable to grow in an optimal growth medium for that organism) was considered the most resistant. Upon this determination, the qualification, validation and operational testing of a medical equipment sterilization device can proceed utilizing only this most resistant organism.

The microorganisms tested were the following: Bacillus subtilis v. niger (ATCC 9372 or 19659), Bacillus stearothermophilus (ATCC 7953), Clostridium sporogenes (ATCC 3584), Staphylococcus aureus (ATCC 6538), Salmonella choleraesuis (ATCC 10708), and Pseudomonas aeruginosa (ATCC 15442). The ideal cell density used was 10⁶, with a minimum of 10⁴ and a maximum of 10⁷. Using a minimum number of three runs the most resistant organism was determined to be Bacillus stearothermophilus.

### Example II

The following describes the protocol for inoculating a household sponge with bacteria, recovery of the bacteria, and tests conducted to reduce bacterial contamination from a household sponge using an air fed ozone generator and a surfactant in a closed volume.

Sample Preparation

A bacteria culture, chosen both for interest as a normal household pathogen and as a pathogenic type of organism found in the food preparation setting, was grown such that preparing an inoculum was consistent. Log phase growth of the bacteria was used because this condition characterized the bacteria causing food spoilage in food left at room temperature for a short period of time or at cold temperature over a longer duration. Bacteria selected were Listeria monocytogenes and Escherichia coli.

The bacteria were grown to log phase and then diluted, or mixed, with phosphate buffered saline water. The bacteria in suspension or inoculum were counted as part of the testing procedure. Bacteria were counted with standard growth media including specialized “agar plates” and 3M Petrifilm (3M Corporation, St. Paul, Minn.). After incubation, colonies of the bacteria were counted and the plate counts were used to calculate the titer of the bacterial suspension.

Phosphate buffered water with bacteria was used to inoculate a sponge sample. This was performed by placing the sponge sample into a sterile sampling bag, adding the bacterial suspension in phosphate buffered water, and then homogenizing the sample using a two-step procedure of blending, or manually massaging, and then allowing the sample to stand for approximately 15 minutes. This method allows the bacteria to be spread throughout the porous sponge surface and/or volume and to adhere to the sponge material. Bacteria will adhere to the sponge material randomly due to hydrophobic interaction.

In addition, a sponge sample was inoculated with a surfactant in the bacterial suspension mixture. In this case, the inoculum was mixed with a surfactant, DAWN™ (Proctor & Gamble Corporation, Cincinnati, Ohio) dish detergent, to a concentration of 1% by volume of DAWN™ dish detergent in the inoculum.

A control procedure was used to compare an ozone treated sponge to a sponge that was not treated. A similarly inoculated sponge was used only for recovery, as follows.

Sample Analysis

The treated or untreated or control sponge was placed into a sterile sampling bag, just as it was inoculated. Phosphate buffered saline water was added to the sampling bag with the sponge. The sample was homogenized similarly to the inoculation procedure. Bacteria were recovered in the phosphate buffered water and then counted on standard growth media to enumerate the recovery titer.

Ozone Treatment

Testing the sponge to eliminate the bacteria utilized treatment inside a gas tight chamber. The chamber contains an ozone-generating electrode that supplies ozone gas inside the unit. Only the air that was present inside the unit supplied the treatment gas. The unit was exhausted of ozone only at the end of the procedure.

The sponge that was inoculated with bacteria and/or the sponge with 1% DAWN™ bacterial suspension was placed inside the chamber. Ozone was produced by a corona discharge from the oxygen in the natural air. Ozone concentrations vary from 100 to 1000 ppm. After a given treatment time period has elapsed, the treatment procedure was stopped. The unit was turned “off” and evacuated. The sponge was collected as one sample.

Treatment Results

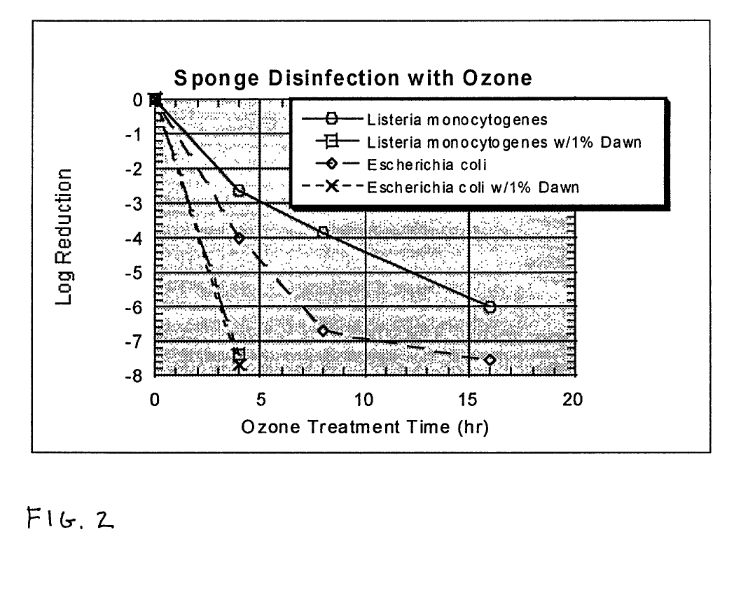

Plate counts of ozone treated and untreated sponge bacteria indicates the efficacy of the method. The results were summarized in the graph (FIG. 2) of the log reduction of colony forming units per milliliter for 4, 8 and 16 hour treatment times, bacteria, and with and without 1% DAWN™. The efficacy of ozone was significantly enhanced with the presence of DAWN™ for the Listeria and E. coli. Four to five log reductions in four hours were observed for ozone treatment with DAWN™ compared to treatment with ozone alone.

References herein to specific Examples or embodiments should not be interpreted as limitations to the invention's scope which is determined by the claims.

What is claimed is:

1. A method of reducing the microbial load on an item comprising porous material, said method comprising:
contacting said porous material with an aqueous solution containing a surfactant, thus wetting the porous material; and
contacting said wetted porous material with a combination of ozone and gas for a period of time sufficient to reduce the microbial load on said item, said gas selected from the group consisting of air, oxygen, and mixtures thereof.

2. The method according to claim 1, wherein the combination of ozone and gas contains from about 1% to about 15% of ozone by weight.

3. The method according to claim 1, wherein the concentration of ozone varies from about 100 ppm to about 1000 ppm.

4. The method according to claim 1, wherein the item is a sponge.

5. The method according to claim 1, wherein the surfactant is a non-cationic surfactant.

6. The method according to claim 5, wherein the surfactant comprises an admixture of biodegradable anionic and cationic surfactants.

7. The method according to claim 1, wherein the microbial load comprises Listeria monocytogenes and Escherichia coli.

8. A method of reducing the microbial load on an item comprising porous material, said method comprising:
contacting said porous material with an aqueous solution containing a surfactant, thus wetting the porous material;
placing said item into a chamber, said chamber having an interior portion in controllable fluid communication with an ozone source, said ozone source actuatable externally to said interior portion;
providing an air tight seal between said interior portion and the atmosphere exterior to said interior portion; and
actuating said ozone source, causing ozone to enter said interior portion, thus contacting said wetted porous material with a combination of ozone and gas for a period of time sufficient to reduce the microbial load on said item, said gas selected from the group consisting of air, oxygen, and mixtures thereof.

9. The method according to claim 8, wherein the concentration of ozone varies from about 100 ppm to about 1000 ppm.

10. The method according to claim 8, wherein the item is a sponge.

11. The method according to claim 8, wherein the surfactant is a non-cationic surfactant.

12. The method according to claim 11, wherein the surfactant comprises an admixture of biodegradable anionic and cationic surfactants.

13. The method according to claim 8, wherein the microbial load comprises Listeria monocytogenes and Escherichia coli.

14. A method of reducing the microbial load on an item comprising porous material, said method comprising:
contacting said porous material with an aqueous solution containing a surfactant, thus wetting the porous material;
placing said item into a chamber, said chamber having an interior portion in controllable fluid communication with an ozone generator capable of generating ozone from the air present in said interior portion, said ozone generator actuatable externally to said interior portion;
providing an airtight seal between said interior portion and atmosphere exterior to said interior portion; and
actuating said ozone generator, thus providing ozone in said interior portion, and contacting said wetted porous material with ozone for a period of time sufficient to reduce the microbial load on said item.

15. The method according to claim 14, wherein the concentration of ozone varies from about 100 ppm to about 1000 ppm.

16. The method according to claim 14, wherein the item is a sponge.

17. The method according to claim 14, wherein the surfactant is a non-cationic surfactant.

18. The method according to claim 17, wherein the surfactant comprises an admixture of biodegradable anionic and cationic surfactants.

19. The method according to claim 14, wherein the microbial load comprises Listeria monocytogenes and Escherichia coli.
